(19) United States
(12) Patent Application Publication
(10) Pub. No.: US 2012/0231033 A1
(43) Pub. Date: Sep. 13, 2012

(54) INACTIVATED POULTRY VACCINE

(76) Inventor: Carla Christina Schrier, Boxmeer (NL)

(21) Appl. No.: 13/510,640

(22) PCT Filed: Nov. 30, 2010
(86) PCT No.: PCT/EP2010/068464

§ 371 Date: May 18, 2012

Related U.S. Application Data
(60) Provisional application No. 61/265,519, filed on Dec. 1, 2009.

(30) Foreign Application Priority Data
Dec. 1, 2009 (EP) 09177634.4

(51) Int. Cl. A61K 39/17; A61K 39/295; A61P 31/14

(52) U.S. Cl. 424/201.1; 424/214.1; 424/202.1

(57) ABSTRACT

The present invention relates to pharmaceutical compositions comprising an immunizing amount of inactivated Newcastle disease and an adjuvant for use in the protection of poultry against Newcastle disease, to the use of an immunogenic amount of an inactivated Newcastle disease virus and an adjuvant for the manufacture of a booster vaccine for the vaccination of a chicken or turkey, and to a kit-of-parts comprises a container comprising an inactivated Newcastle disease virus and an adjuvant for a priming vaccination of a chicken or turkey and a container comprising an inactivated Newcastle disease virus and an adjuvant for a boosting vaccination of a chicken or turkey.

The present invention relates to pharmaceutical compositions comprising an immunizing amount of inactivated Newcastle disease and an adjuvant for use in the protection of poultry against Newcastle disease, to the use of an immunogenic amount of an inactivated Newcastle disease virus and an adjuvant for the manufacture of a booster vaccine for the vaccination of a chicken or turkey, and to a kit-of-parts comprises a container comprising an inactivated Newcastle disease virus and an adjuvant for a priming vaccination of a chicken or turkey and a container comprising an inactivated Newcastle disease virus and an adjuvant for a boosting vaccination of a chicken or turkey.

Commercial poultry farming can roughly be divided in three specializations: the production of layer chickens, breeders and broilers.

Both broilers, layers and breeders are vulnerable to many viral, bacterial and parasitic infections. As a measure of precaution, they are vaccinated against i.a. many viral diseases already in an early stage of their life.

One of the virus vaccines that is part of the standard vaccination scheme of by far most chicken or turkey farmers is a Newcastle disease virus vaccine. Newcastle disease is a viral infection of poultry with a wide geographical distribution causing great economic losses in the poultry industry. Newcastle disease virus (NDV) is the etiologic agent of this disease and represents the prototype virus of the genus Paramyxovirus. Newcastle disease is complicated in that different isolates and strains of the virus may induce enormous variation in the severity of the disease. In general, the younger the chicken or turkey the more acute and severe the disease. The infection may take place by either inhalation or ingestion and the infectious form of the virus is spread from one bird to another.

As mentioned-above, several pathotypes of NDV have been identified, i.e. velogenic, mesogenic and lentogenic. The neurotropic velogenic form of disease is caused by highly pathogenic strains of NDV and is characterised by a sudden onset of severe respiratory signs followed by neurological signs. In most cases the infected animals do not survive. Viscerotropic velogenic NDV strains are highly pathogenic and cause high mortality and severe lesions in the gastrointestinal tract. Mesogenic strains of NDV usually cause severe respiratory disease in fully susceptible birds, and both mesogenic and velogenic strains cause a marked drop in egg production in adult birds. Lentogenic strains of ND virus cause generally a mild disease which is characterised by respiratory signs, especially in young fully susceptible birds.

In order to reduce the economic losses due to ND in the commercial poultry industry chickens have been vaccinated against ND. Live vaccines derived from lentogenic and mesogenic strains have been applied routinely, the mesogenic vaccine being suitable only for secondary vaccination. However, also in the lentogenic group there is a considerable range in virulence. NDV strains used as live vaccines include V4, Hitchner B1, F, La Sota (lentogenic), and strain H, Mukteswar, Komarov and Roakin (mesogenic).

As a rule of thumb, broilers have a life span of only 1-12 weeks. Therefore, they are mainly vaccinated with live attenuated NDV vaccines.

Breeders and broilers have a significantly longer life span and it is important to have them protected against infectious diseases through their whole life span. This means that they would even more benefit from vaccines providing an early and long lasting protection.

In the early days of poultry vaccination, live attenuated vaccines were either unsafe or non-existing. Therefore, between 1950 and roughly 1960, chickens had to be vaccinated with inactivated virus vaccines.

Such vaccines were however administered without any adjuvants or with the only adjuvantia available at that time; adjuvantia with severe, nowadays unacceptable, side effects.

No really acceptable adjuvantia were available then.

Equally, the oil-in-water emulsions and water-in-oil emulsions currently in use were unknown in that period.

Over the years however, safe live vaccines for the protection of poultry were developed. The main advantage of live ND vaccines is that these can be administered by inexpensive mass application techniques, such as spray and drinking water application.

Live vaccines further have as advantages that they are less expensive per dose and provide relatively immediate protection. Moreover, they closely mimic the natural infection.

And finally, live attenuated ND vaccines, when administered through application techniques such as spray and drinking water application, provide a good local protection in addition to systemic protection.

Therefore, in modern poultry farming live attenuated Newcastle disease vaccines are the vaccines used for the prime vaccination of chickens or turkeys.

A disadvantage of priming with live vaccinated vaccines is, that their protection is not really long-lasting, so a second and possibly a third vaccination with live attenuated vaccines must be given when necessary.

However, over the years it was also found that inactivated vaccines, when given as a booster vaccine after primer vaccination with a live attenuated vaccine, have the advantage that they provide a longer lasting immunity in chickens or turkeys that were primed with a live attenuated vaccines.

Therefore nowadays the second, so-called booster, vaccination is therefore preferably given with an inactivated vaccine.

Already many years ago, such vaccination schemes combining live attenuated and inactivated vaccines have been proven to be highly effective (Glisson, J. R. and Kleven, S. H. 1993 Poultry vaccines. In: Peters, A. R. (Ed.), Vaccines for Veterinary Applications. Butterworth Heinemann, Oxford, UK, pp. 170-173 (1993)).

As a consequence, the current practice in poultry vaccination is as follows: for Newcastle disease, and in fact for many other viral poultry diseases such as infectious bronchitis, live attenuated virus vaccines are used for primer vaccination, whereas a booster with inactivated vaccines is used to induce a prolonged antibody response in birds that were primed with live attenuated virus vaccines. See e.g. “Vaccination Strategies”, Glisson, J. R., in Poultry Digest page 12-16, December 1999/January 2000.

Hilgers, L. A. Th., et al., have once more underlined the importance of the current standard vaccination therapy: priming with a live vaccine and boosting with an inactivated vaccine, for e.g. Newcastle disease and Infectious Bronchitis in chickens. In this paper they checked the effects of various adjuvants given with the inactivated booster vaccine. (Vet. Immunol. and Immunopath. 66:159-171 (1998).

One of the few disadvantages in the use of live attenuated vaccines is that, while inducing local immunity, they may cause significant vaccination reactions, in particular in the respiratory tract after spray vaccination.

This unavoidable disadvantage is up till now to a certain extend dealt with by using very mild/attenuated viruses for vaccination, in particular for primary vaccination in young animals.

It is an objective of the present invention to provide ways to solve this problem in an alternative way.

It was surprisingly found now, that efficacious vaccination by means of both priming and boosting with an inactivated vaccine is feasible, provided that both prime and boost vaccine comprise a suitable adjuvant.

Even more surprisingly, it was found that an inactivated NDV vaccine prime/boost regime not only provides excellent systemic protection, but also provides protection of the trachea. This is highly unexpected since inactivated vaccines have to be administered through the parenteral route, so no induction of local immunity in the trachea is to be expected at all.

A clear advantage of this surprising effect is, that animals vaccinated according to this regime are hardly affected, if at all, by field infection. This is both beneficial to the animal and the farmer. And of course the most important advantage is that the risks involved in using live attenuated viruses in very young animals can be fully avoided.

The inactivated NDV prime/boost vaccination regime can be used in broilers, breeders and layer stock. For broilers, prime vaccination is preferably done as soon as possible. Preferably, priming is done between 1 and 7 days of age. A boost vaccination is preferably given between 2 and 12 weeks after the priming. In regions with a high NDV-infection pressure, a second vaccination may be done at 2 weeks after the priming. A more standard vaccination scheme would have 4-10 weeks between priming and boosting.

As mentioned above, depending on the production methods used in different countries, in many cases broilers do not reach the age of 10 weeks.

Thus, a preferred vaccination regime would be the following: the booster would be given about 4-8 weeks after the priming, and in cases where broilers are slaughtered after 5-6 weeks, a booster would be given at about 4 weeks. The priming vaccination would consequently be given between 1-14 days in such cases.

For breeders and layers, a prime vaccination is also preferably given between 1 and 7 days of age, and a boost vaccination can be given from 2 to 12 weeks after the priming In this case, although depending on the infection pressure, booster vaccination could be postponed until 12 weeks, in order to extend the duration of immunity with a few more weeks.

Generally spoken, however, the preferred time span between priming and boosting is also between 4 and 8 weeks.

A first embodiment of the present invention thus relates to the use of an immunogenic amount of an inactivated Newcastle disease virus and an adjuvant for the manufacture of a booster vaccine for the vaccination of a chicken or turkey that has been vaccinated with a priming vaccine comprising an immunogenic amount of inactivated Newcastle disease virus and an adjuvant, between 2 and 12 weeks prior to being vaccinated with the booster vaccine.

Methods for inactivation of Newcastle Disease virus are well-known in the art. Chemical inactivation can be done e.g. with well-known inactivating compounds such as formaldehyde, beta-propiolactone and bi-ethylene-imine. Physical inactivation can be done e.g. by heat inactivation or by irradiation with X- or gamma-radiation, or with UV-light.

The amount of inactivated NDV antigen in the vaccine can in principle be between 10² and 10¹⁰ equivalents of egg infectious doses (EID₅₀). (Equivalent, because in the vaccine the viruses would be inactivated and thus not be egg infective). In practice however, 10² EID₅₀ would be considered a low dose, that would only be efficacious in the presence of a very strong adjuvant. An amount of between 10⁴ and 10⁸ would be a preferred dose. A dose of 10¹⁰ EID₅₀, although suitable, would be unattractive from an economical point of view.

There are many adjuvants known in the art that are suitable in combination with inactivated NDV, such as alum hydroxide, alumin phosphate, saponins, vegetable oils such as tocopherol and mineral oils. Very efficient adjuvants are oil-in-water emulsions and especially water-in-oil emulsions, further also referred to as are oil-in-water adjuvants and water-in-oil adjuvants. Such emulsions are well-known in the art. Thus, preferably, the booster vaccine or pharmaceutical compound for use according to the invention comprises a water-in-oil adjuvant.

The route of administration of the inactivated vaccine is in principle the parenteral route. Administration can be done e.g. intramuscular or subcutaneous. These routes of administration and their particulars are well-known in the art of poultry vaccination.

The inactivated NDV prime-boost vaccination regime is equally suitable for chickens and turkeys.

Another embodiment of the present invention relates to a pharmaceutical composition comprising an immunogenic amount of inactivated Newcastle disease virus and an adjuvant, for use in the treatment of Newcastle disease in a chicken or turkey, wherein said treatment comprises the steps of administering a priming vaccination to said chicken or turkey with a vaccine comprising an immunogenic amount of inactivated Newcastle disease virus and an adjuvant, and administering a boosting vaccination within 2-12 weeks of said priming vaccination, to said chicken or turkey with a vaccine comprising an immunogenic amount of inactivated Newcastle disease virus and an adjuvant.

Preferably, the priming and/or booster vaccine or the pharmaceutical composition comprise in addition to the NDV component defined above, one or more additional antigens derived from a virus or micro-organism pathogenic to poultry or genetic information encoding said antigen.

More preferably, the virus or micro-organism is selected from the group consisting of Infectious Bronchitis virus, Infectious Bursal Disease (Gumboro), Chicken Anaemia agent, Avian Reovirus, Mycoplasma gallisepticum, Turkey Rhinotracheitis virus, Haemophilus paragallinarum (Coryza), Chicken Poxvirus, Avian Encephalomyelitis virus, Egg Drop syndrome virus, Infectious Laryngotracheitis virus, Herpes Virus of Turkeys, Eimeria species, Ornithobacterium rhinotracheale, Pasteurella multocida, Mycoplasma synoviae, Salmonella species and E. coli.

The priming and booster vaccine and the pharmaceutical composition can be prepared and marketed in the form of a suspension, or in a lyophilized form. They will additionally contain a pharmaceutically acceptable carrier customarily used for such active components. Carriers can be, or may include, stabilizers, diluents, preservatives and buffers.

Suitable stabilizers are for example SPGA, carbohydrates (such as dried milk, serum albumin or casein) or degradation products thereof. Suitable buffers are for example alkali metal phosphates. Suitable preservatives are thimerosal, merthiolate and gentamicin. Diluents include water, aqueous buffers (such as buffered saline), alcohols and polyols (such as glycerol).

Preferably, the vaccines and pharmaceutical compositions are freeze-dried, in order to make them less dependent on cold storage.

An immunogenic amount of NDV is the amount of NDV that induces an immune response in chickens or turkeys that decreases the pathological effects of the disease, when compared to the pathological effects after infection with a wild-type NDV in non-immunized birds.

ND virus strains suitable for the preparation of inactivated NDV vaccines, more specifically oil-emulsion vaccines, include such strains as Clone 30, Ulster 2C, Hitchner B1, La Sota, Roakin and various virulent viruses (D. J. Alexander, In Diseases of Poultry, 9th edition 1991, eds. Calnek et al., Iowa State University Press, Ames, Iowa, 496-519).

Another embodiment of the present invention relates to a kit of parts, said kit comprising a container comprising an inactivated Newcastle disease virus and an adjuvant for a priming vaccination of a chicken or turkey and a container comprising an inactivated Newcastle disease virus and an adjuvant for a boosting vaccination of a chicken or turkey. A container in this respect can be e.g. a classical vaccine flask or a tube or any other object, of any material, in which a vaccine can be stored.

### EXAMPLES

### Example 1

The aim of this experiment is to assess whether a prime-boost vaccination strategy with inactivated lentogenic NDV strain formulated in GNE induces local protection against a locally given live mesogenic NDV strain Beaudette challenge. (GNE is a standard water-in-oil emulsion on the basis of mineral oil).

### 1 Materials and Methods

### 1.1 Set Up of the Experiment

One (1) group of 3-4 weeks-old SPF White Leghorn chickens (group 1), placed in isolators, was vaccinated twice intramuscularly (i.m.) in the breast muscle with a water-in-oil emulsion vaccine containing approximately 10̂7.7 EID50 of a formaldehyde inactivated lentogenic NDV strain per dose as indicated in Table 1. Group 2 was not vaccinated. At the beginning of the experiment, and 6 and 8 weeks post primo vaccination blood was taken from all animals from both groups. Eight weeks post primo vaccination chickens from both groups were challenged via the oculo route with NDV Beaudette. Two swabs at 3, 4 and 7 days post-challenge were taken from the oropharynx and cloaca and used for virus re-isolation. Fifteen days post challenge blood was taken from all animals from both groups.

TABLE 1
Grouping and dosing

| gr | N | inoculum | Challenge | isolator |
|---|---|---|---|---|
| 1. | 10 | 10\hat{ }7.7 EID50 | NDV Beaudette | A13 |
| | | formaldehyde | | |
| | | inactivated lentogenic | | |
| | | NDV strain per dose, in | | |
| | | w/o emulsion | | |
| 2. | 10 | — | NDV Beaudette | A14 |

### 1.2 Vaccination

Primo vaccination: Only the chickens from group 1 received 10̂7.7 EID50 formaldehyde inactivated lentogenic NDV strain per dose, in w/o emulsion, given i.m. in the breast muscle at the age of 3-4 weeks-old.

Booster vaccination: Only the animals from group 1 received 10̂7.7 EID50 formaldehyde inactivated lentogenic NDV strain per dose, in w/o emulsion, given i.m. in the breast muscle 6 weeks after the primo vaccination.

### 1.3 Challenge

Eight weeks post primo vaccination all animals from both groups were challenged via the oculo route with Live NDV Beaudette (10^{6.0} EID₅₀ per animal; 100 μl in each eye via eye drop).

### 1.4 Blood Samples

Blood samples for serology were taken at 0, 6 and 8 weeks post primo-vaccination, and at 15 days post challenge from all chickens from both groups.

### 1.5 Swabs

At 2, 3, 4 and 7 days post challenge swabs from the oropharynx and cloaca were taken from all chickens for virus re-isolation. Swabs were collected in 2.5 ml of Tryptose 2.5% to which 1000 U/1000 μg per ml Pen/Strep was added.

### 1.6 Observation for Clinical Signs

For a period of 15 days after challenge, chickens were scored daily for the occurrence of clinical evidence of NDV Beaudette infection or mortality. Data were recorded on a form as outlined in Addendum 1. The following score system was used:
0: healthy
1: watery eyes—coughing
2: respiratory distress
3: dead

### 1.7 HI-Assay

Serum levels of NDV-specific antibodies were determined by a haemagglutination-inhibition (HI) assay. Serial two-fold dilutions of sera were prepared in microtiter plates and mixed with an equal volume containing 8 haemagglutinating units/50 μl NDV antigen. After at least 30 minutes of incubation, chicken red blood cells were added. Titres are expressed as the reciprocal of the highest dilution that gives complete inhibition of haemagglutination of red blood cells (1% (v/v) in buffered saline). Samples were regarded positive for inhibition of haemagglutination at a dilution≧1:2.

### 1.8 Virus Re-Isolation

Re-isolation of virus was performed by inoculation of 10-day-old embryonated chicken eggs (N=8) with 0.1 ml of undiluted sample material. Following incubation for 4-6 days the allantoic fluid from all eggs was tested for the presence of NDV by determination of the HA-activity. The titer was calculated according to the method of Spaerman and Kaerber (In: B. Bibrack and G. Whittmann, Editors, Virologische arbeitsmethoden, Fisher Verlag, Stuttgart (1974), pp. 37-39).

### 2 Results

### 2.1 Observation for Clinical Signs

The observation showed that none of the vaccinated birds (group 1) had clinical signs. Only 1 bird from the non-vaccinated challenged group (group 2) had a score of 1, i.e. watery eyes—coughing, starting at 10 days post challenge till the end of the observation period of 15 days. None of the birds died.

### 2.2 HI-Titers

The HI-titers were measured using the sera from time points 6 and 8 weeks post primo vaccination and from 15 days post challenge (see Table 2).

TABLE 2
NDV HI-titers

| | | mean log2 HI antibody titre (std) | | |
|---|---|---|---|---|
| | | 6 wks | 2 wks | 2 wks |
| gr | Inoculum | post primo | post boost | post challenge |
| 1. | 10\hat{ }7.7 EID50 | 6.0 (1.5) | 6.7 (1.3) | 10.3 (1.2) |
| | formaldehyde | | | |
| | inactivated | | | |
| | lentogenic NDV | | | |
| | strain per dose, in | | | |
| | w/o emulsion | | | |
| 2. | — | 0.6 (0.5) | 0.9 (0.3) | 10.6 (0.7) |

### 2.3 Virus Re-Isolation

Re-isolation of NDV Beaudette virus was performed using oropharyngal and cloacal swabs taken at 3, 4 and 7 days post challenge and are summarized in Table 3.

TABLE 3
Virus re-isolation

| | | percentage positive swabs taken from oropharynx or | | | | | |
|---|---|---|---|---|---|---|---|
| | | cloaca at the indicated time points post challenge | | | | | |
| | | oropharyngal swabs | | | cloacal swabs | | |
| gr | Inoculum | 3 days | 4 days | 7 days | 3 days | 4 days | 7 days |
| 1. | 10\hat{ }7.7 | 50% | 100% | 40% | 0% | 0% | 0% |
| | EID50 | (5/10) | (10/10) | (4/10) | (0/10) | (0/10) | (0/10) |
| | formal- | | | | | | |
| | dehyde | | | | | | |
| | inactivated | | | | | | |
| | lentogenic | | | | | | |
| | NDV | | | | | | |
| | strain per | | | | | | |
| | dose, | | | | | | |
| | in w/o | | | | | | |
| | emulsion | | | | | | |
| 2. | — | 80% | 100% | 100% | 30% | 100% | 100% |
| | | (8/10) | (10/10) | (10/10) | (3/10) | (10/10) | (10/10) |

TABLE 4
Percentage protection based on virus re-isolation (Table 3)

| | | swabs are taken at the indicated time points post | | | | | |
|---|---|---|---|---|---|---|---|
| | | challenge | | | | | |
| | | oropharyngal swabs | | | cloacal swabs | | |
| gr | inoculum | 3 days | 4 days | 7 days | 3 days | 4 days | 7 days |
| 1 | 10\hat{ }7.7 | 50% | 0% | 60% | 100% | 100% | 100% |
| | EID50 | (5/10) | (0/10) | (6/10) | (10/10) | (10/10) | (10/10) |
| | formal- | | | | | | |
| | dehyde | | | | | | |
| | inactivated | | | | | | |
| | lentogenic | | | | | | |
| | NDV | | | | | | |
| | strain | | | | | | |
| | per dose, | | | | | | |
| | in w/o | | | | | | |
| | emulsion | | | | | | |
| 2. | — | 20% | 0% | 0% | 70% | 0% | 0% |
| | | (2/10) | (0/10) | (0/10) | (7/10) | (0/10) | (0/10) |

The results show that non-vaccinated challenged control animals are not protected against NDV Beaudette at 4 and 7 days post challenge (see Table 4), as the virus could be re-isolated at these time points (see Table 3). At 3 days post challenge only 20% and 70% of the birds are protected based on virus re-isolation from oropharyngal and cloacal swabs, respectively.

In none of the cloacal swab samples taken from the vaccinated challenged birds at 3, 4 and 7 days post challenge virus could be isolated (see Table 3) indicating that all of these birds (100%) are protected (see Table 4). From the oropharyngal swabs 50% (5/10), 100% (10/10) and 40% (4/10) were virus positive at 3, 4 and 7 days post challenge, respectively (see Table 3).

### Discussion

The prime-boost vaccination regime with inactivated NDV formulated in GNE induces sufficient protection to reduce the spreading of, via the ocular route given, NDV Beaudette as indicated by the decreased percentage of positive oropharyngal swabs at 7 days post challenge. From cloacal swabs taken from the same vaccinated birds no virus could be re-isolated at any time point indicating that these animals are protected.

In summary; a prime-boost strategy with inactivated NDV formulated in a w/o emulsion induces sufficient (local) immunological protection to protect chickens against the intermediate virulent NDV Beaudette virus.

### Example 2

The aim of this experiment is, to assess whether a prime and boost vaccination strategy with inactivated NDV formulated in GNE induces systemic immunity against a systemically given live NDV Herts 33/56 challenge.

### 1 Materials and Methods

### 1.1 Set Up of the Experiment

Ten 3-4 weeks-old SPF White Leghorn chickens (Lohmann), from a group of 12 animals placed in an isolator, were vaccinated twice intramuscularly (i.m.) in the breast muscle with 10̂7.7 EID50 formaldehyde inactivated lentogenic NDV strain per dose, in w/o emulsion, as indicated in Table 1. The other 2 animals were not vaccinated. At the beginning of the experiment, and 6 and 8 weeks post primo vaccination blood was taken from all animals. Eight weeks post primo vaccination all chickens were challenged via the i.m. route with 0.2 ml (10^{6.0} EID₅₀) of the live velogenic NDV Herts 33/56 strain. Two swabs at 3, 4 and 7 days post-challenge were taken from the oropharynx (throat) and cloaca and used for virus re-isolation. Fifteen days post challenge (p.c.) a final blood sample was taken from all animals.

TABLE 1
Grouping and dosing

| gr | N | IP nr | antigen | adjuvant | challenge | isolator |
|---|---|---|---|---|---|---|
| 1. | 10 | B290A | inact. NDV | GNE | NDV Herts | A12 |
| 2. | 2 | — | — | — | NDV Herts | |

### 1.2 Vaccination

Primo vaccination: Ten out of 12 chickens from group 1 received 0.5 ml vaccine i.m. in the breast muscle at the age of 3-4 weeks.

Booster vaccination: The same 10 animals from the primo vaccination received 0.5 ml vaccine i.m. in the breast muscle 6 weeks after the primo vaccination.

### 1.3 Challenge

Eight weeks post primo vaccination all animals were challenged with 0.2 ml live velogenic NDV Herts 33/56 (10^{6.0} EID₅₀ per chicken) via the i.m. route in the leg muscle.

### 1.4 Blood Samples

Blood samples for serology were taken at 0, 6 and 8 weeks post primo-vaccination, and at 15 days post challenge from all chickens from both groups.

### 1.5 Swabs

At 2, 3, 4 and 7 days post challenge swabs from the oropharynx and cloaca were taken from all chickens for virus re-isolation. Swabs were each collected in 2.5 ml of Tryptose 2.5% to which 1000 U/1000 μg per ml Pen/Strep was added.

### 1.6 Observation for Clinical Signs

For a period of 15 days after challenge, chickens were scored daily for the occurrence of clinical evidence of NDV infection or mortality. The following score system was used:
0: No occurrence of clinical evidence of Newcastle disease.
1: Occurrence of clinical evidence of Newcastle disease
   Central nervous signs like:
      Clonic spasm
      Muscular tremors
      Torticollis
      Opisthotonos
      Paralysis of legs or wings
2: Mortality caused by NDV challenge.

### 1.7 HI-Assay

Serum levels of NDV-specific antibodies were determined by a haemagglutination-inhibition (HI) assay. Serial two-fold dilutions of sera were prepared in microtiter plates and mixed with an equal volume containing 8 haemagglutinating units/50 μl NDV antigen. After at least 30 minutes of incubation, chicken red blood cells were added. Titres are expressed as the reciprocal of the highest dilution that gives complete inhibition of haemagglutination of red blood cells (1% (v/v) in buffered saline). Samples were regarded positive for inhibition of haemagglutination at a dilution≧1:2.

### 1.8 Virus Re-Isolation

Re-isolation of virus was performed by inoculation of 10-day-old embryonated eggs (N=8) with 0.1 ml of undiluted sample material. Following incubation for 4-6 days the allantoic fluid from all eggs was tested for the presence of NDV by determination of the HA-activity. The titer was calculated according to the method of Spaerman and Kaerber (In: B. Bibrack and G. Whittmann, Editors, Virologische arbeitsmethoden, Fisher Verlag, Stuttgart (1974), pp. 37-39).

### 2 Results

### 2.1 Observation for Clinical Signs

The observation showed that none of the 10 vaccinated birds had clinical signs. One of the 2 non-vaccinated birds had a score of 1, i.e. occurrence of clinical evidence of Newcastle disease, 3 days post challenge, and a score of 2, i.e. mortality caused by NDV challenge, at day 4 post challenge. The other bird that was not vaccinated had a score of 1 three days post challenge, and a score of 2 in the afternoon of the same day.

### 2.2 HI-Titers

The HI-titers were measured using the sera from time points 6 and 8 weeks post primo vaccination and from 15 days post challenge (see Table 2).

TABLE 2
Anti-NDV HI-titers

| | | mean log2 HI antibody titre (std) | | |
|---|---|---|---|---|
| | | 6 wks | 2 wks | 2 wks |
| gr | inoculum | post primo | post boost | post challenge |
| 1. | 10\hat{ }7.7 EID50 | 5.0 (2.3) | 5.7 (2.1) | 8.9 (1.2) |
| | formaldehyde | | | |
| | inactivated | | | |
| | lentogenic NDV | | | |
| | strain per dose, in | | | |
| | w/o emulsion | | | |
| 2. | — | 0.0 (0.5) | 0.0 (0.3) | — |

### 2.3 Virus Re-Isolation

Re-isolation of NDV Herts virus was performed using oropharyngal and cloacal swabs taken at 3, 4 and 7 days post challenge and are summarized in Table 3.

TABLE 3
Virus re-isolation

| | | percentage positive swabs taken from oropharynx or | | | | | |
|---|---|---|---|---|---|---|---|
| | | cloaca at the indicated time points post challenge | | | | | |
| | | oropharyngal swabs | | | cloacal swabs | | |
| gr | inoculum | 3 days | 4 days | 7 days | 3 days | 4 days | 7 days |
| 1. | 10\hat{ }7.7 | 20% | 20% | 10% | 20% | 30% | 0% |
| | EID50 | (2/10) | (2/10) | (1/10) | (2/10) | (30/10) | (0/10) |
| | formal- | | | | | | |
| | dehyde | | | | | | |
| | inactivated | | | | | | |
| | lentogenic | | | | | | |
| | NDV | | | | | | |
| | strain | | | | | | |
| | per dose, | | | | | | |
| | in w/o | | | | | | |
| | emulsion | | | | | | |
| 2. | — | — | — | — | — | — | — |

TABLE 4
Percentage protection based on mortality (Table 3)

| | | swabs are taken at the indicated time points post | | | | | |
|---|---|---|---|---|---|---|---|
| | | challenge | | | | | |
| | | oropharyngal swabs | | | cloacal swabs | | |
| gr | inoculum | 3 days | 4 days | 7 days | 3 days | 4 days | 7 days |
| 1. | 10\hat{ }7.7 | 100% | 100% | 100% | 100% | 100% | 100% |
| | EID50 | | | | | | |
| | formal- | | | | | | |
| | dehyde | | | | | | |
| | inactivated | | | | | | |
| | lentogenic | | | | | | |
| | NDV | | | | | | |
| | strain | | | | | | |
| | per dose, | | | | | | |
| | in w/o | | | | | | |
| | emulsion | | | | | | |
| 2. | — | 0% | 0% | 0% | 0% | 0% | 0% |

The results show that the non-vaccinated challenged control animals are not protected against NDV Herts.

All vaccinated challenged birds are protected against NDV Herts.

### Discussion

The prime-boost vaccination regime with inactivated NDV formulated in a w/o emulsion induces full systemic protection to protect chickens against the virulent NDV Herts virus.

1-5. (canceled)

6. A pharmaceutical composition for use in the treatment of Newcastle disease in a chicken or turkey comprising an immunogenic amount of inactivated Newcastle disease virus and a water-in-oil adjuvant.

7. The pharmaceutical composition of claim 6, characterised in that said composition comprises one or more additional antigens derived from a virus or micro-organism pathogenic to poultry, or genetic information encoding said additional antigen.

8. The pharmaceutical composition of claim 7, characterized in that said virus or micro-organism is selected from the group consisting of Infectious Bronchitis virus, Infectious Bursal Disease (Gumboro), Chicken Anaemia agent, Avian Reovirus, Mycoplasma gallisepticum, Turkey Rhinotracheitis virus, Haemophilus paragallinarum (Coryza), Chicken Poxvirus, Avian Encephalomyelitis virus, Egg Drop syndrome virus, Infectious Laryngotracheitis virus, Herpes Virus of Turkeys, Eimeria species, Ornithobacterium rhinotracheale, Pasteurella multocida, Mycoplasma synoviae, Salmonella species and E. coli.

9. A kit comprising:
(i) a container that comprises an inactivated Newcastle disease virus and an adjuvant for a priming vaccination of a chicken or turkey; and
(ii) a container comprising an inactivated Newcastle disease virus and an adjuvant for a boosting vaccination of a chicken or turkey.

10. The kit of claim 9 wherein said adjuvant for the priming vaccination is a water-in-oil adjuvant, and wherein said adjuvant for the boosting vaccination is a water-in-oil adjuvant.

11. A method of treating Newcastle disease in a chicken or turkey, comprising:
(i) administering to said chicken or turkey a priming vaccine comprising an immunogenic amount of inactivated Newcastle disease virus and a water-in-oil adjuvant; and
(ii) administering to said chicken or turkey a boosting vaccine within 2-12 weeks of said priming vaccination; wherein the boosting vaccine comprises an immunogenic amount of inactivated Newcastle disease virus and a water-in-oil adjuvant.

12. The method of claim 11 wherein the priming vaccine further comprises one or more additional antigens derived from a virus or micro-organism pathogenic to poultry, or genetic information encoding said additional antigen.

13. The method of claim 12 wherein said virus or micro-organism is selected from the group consisting of Infectious Bronchitis virus, Infectious Bursal Disease (Gumboro), Chicken Anaemia agent, Avian Reovirus, Mycoplasma gallisepticum, Turkey Rhinotracheitis virus, Haemophilus paragallinarum (Coryza), Chicken Poxvirus, Avian Encephalomyelitis virus, Egg Drop syndrome virus, Infectious Laryngotracheitis virus, Herpes Virus of Turkeys, Eimeria species, Ornithobacterium rhinotracheale, Pasteurella multocida, Mycoplasma synoviae, Salmonella species and E. coli.

14. The method of claim 11 wherein the boosting vaccine further comprises one or more additional antigens derived from a virus or micro-organism pathogenic to poultry, or genetic information encoding said additional antigen.

15. The method of claim 14 wherein said virus or micro-organism is selected from the group consisting of Infectious Bronchitis virus, Infectious Bursal Disease (Gumboro), Chicken Anaemia agent, Avian Reovirus, Mycoplasma gallisepticum, Turkey Rhinotracheitis virus, Haemophilus paragallinarum (Coryza), Chicken Poxvirus, Avian Encephalomyelitis virus, Egg Drop syndrome virus, Infectious Laryngotracheitis virus, Herpes Virus of Turkeys, Eimeria species, Ornithobacterium rhinotracheale, Pasteurella multocida, Mycoplasma synoviae, Salmonella species and E. coli.

16. The method of claim 12 wherein the boosting vaccine further comprises one or more additional antigens derived from a virus or micro-organism pathogenic to poultry, or genetic information encoding said additional antigen.

17. The method of claim 16 wherein said virus or micro-organism of the boosting vaccine is selected from the group consisting of Infectious Bronchitis virus, Infectious Bursal Disease (Gumboro), Chicken Anaemia agent, Avian Reovirus, Mycoplasma gallisepticum, Turkey Rhinotracheitis virus, Haemophilus paragallinarum (Coryza), Chicken Poxvirus, Avian Encephalomyelitis virus, Egg Drop syndrome virus, Infectious Laryngotracheitis virus, Herpes Virus of Turkeys, Eimeria species, Ornithobacterium rhinotracheale, Pasteurella multocida, Mycoplasma synoviae, Salmonella species and E. coli.
